(19) United States
(12) Patent Application Publication
(10) Pub. No.: US 2003/0199024 A1
(43) Pub. Date: Oct. 23, 2003

(54) Single chain trimers of class I MHC molecules

(76) Inventor: Ted H. Hansen, St. Louis, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/126,335

(22) Filed: Apr. 19, 2002

(51) Int. Cl. C12P 21/02; C07K 14/74; C07K 16/28; C07H 21/04; C12N 5/06

(52) U.S. Cl. 435/69.1; 435/320.1; 435/326; 530/388.22; 536/23.53; 530/350; 435/252.3; 536/23.5; 435/325

(57) ABSTRACT

A recombinant DNA molecule is comprised of a DNA sequence that encodes a single chain trimer of a novel mature class I MHC molecule. The single chain trimer contains, in sequence from the N-terminus to the C-terminus: a peptide ligand segment; (2) a first linker; (3) a β₂m segment; (4) a second linker; and (5) a class I heavy chain segment, wherein the peptide ligand segment has a carboxy end, the β₂m segment has amino and carboxy ends, and the heavy chain segment has an amino end, and wherein the peptide ligand segment is covalently linked via its carboxy end to the amino end of the β₂m segment by the first linker, and wherein the β₂m segment is covalently linked via its carboxy end to the amino end of the heavy chain segment by the second linker.

### GOVERNMENT RIGHTS IN THE INVENTION

This invention was made with the support of Government Grants AI19687, AI42793 and AI46553 from the National Institutes of Health. The government of the United States of America has certain rights in this invention.

### BACKGROUND OF THE INVENTION

Throughout this application various publications are referenced. The disclosures of these publications in their entireties are hereby incorporated by reference in this application in order to more fully describe the state of the art to which this invention pertains.

1. Field of the Invention

This invention relates to the biochemical arts. More particularly it relates to complexes of major histocompatibility complex (MHC) molecules.

2. Discussion of the Related Art

Antigen-specific T cell responses are invoked by antigenic peptides bound to the binding groove or cleft of major histocompatibility complex (MHC) glycoproteins as part of the mechanism of the immune system to identify and respond to foreign antigens. The bound antigenic peptides interact with T cell receptors and thereby modulate an immune response. The antigenic peptides are bound by non-covalent means to particular “binding pockets” comprised of polymorphic residues of the MHC protein's binding groove.

The glycoproteins encoded by the MHC have been extensively studied in both the human and murine systems. In general, they have been classified as Class I glycoproteins, found on the surfaces of all cells and primarily recognized by cytotoxic T cells; and Class II glycoproteins which are found on the surfaces of several cells, including accessory cells such as macrophages, and are involved in presentation of antigens to helper T cells. Many of the histocompatibility proteins have been isolated and characterized. For a general review of MHC glycoprotein structure and function, see Fundamental Immunology, 2d Ed., W. E. Paul, ed., Ravens Press N.Y. 1989.

The class I genes (HLA-A, B and C in humans, H-2K, D, and L in mice) code for multi-determinant antigens which appear on the surface of cells are comprised of heavy and light peptide chains. Only the heavy chain is encoded by the MHC. It contains hypervariable regions analogous to the immunoglobulins. The heavy chain consists of a large transmembrane glycoprotein of about 44K molecular weight (350 amino acids). This heavy chain is non-covalently associated with the light chain, beta-2-microglobulin (β₂m), an 100 amino acid, 12K molecular weight protein. β₂m is encoded by genes on a separate chromosome than those coding for the class I heavy chains.

Class I heavy chains require full assembly with β₂m and a high affinity peptide to be stably expressed as class I MHC molecules at the cell surface at levels sufficient to induce optimal T cell immunity (Townsend et al., 1990). Cells from mice deficient in β₂m (Zijlstra et al., 1990; Koller et al., 1990) or high affinity peptide (Van Kaer et al., 1992) express few class I MHC molecules at the cell surface. Instead, the preponderance of incompletely assembled class I s accumulate in the ER and are targeted for degradation (Raposo et al., 1995).

Pathogens and tumors have developed elaborate mechanisms to block class I MHC assembly as a means of evading immune detection (Ploegh, 1998; Miller and Sedmak, 1999; Hengel et al 1998; Seliger et al. 2000). For example, progressively growing tumor cells are frequently found to have reduced class I MHC expression caused by β₂M-deficiency or TAP deficiency (Seliger et al. 2000). In addition, viruses have evolved elaborate mechanisms to prevent TAP-mediated peptide transport with viral proteins such as herpes simplex virus protein ICP47 (York et al. 1996: Fruh et al., 1995; Hill et al., 1995) or human cytomegalovirus protein US6 (Ahn et al., 1997). Furthermore, other viral proteins such as adenovirus protein E19 have been reported to interfere with class I MHC assembly by blocking its interaction with tapasin, thus preventing TAP association (Bennett et al., 1999). Similarly, viruses and tumors may block the interaction of class I MHC with other ER chaperones as a means to impair full assembly of class I MHC and thus reduce levels of surface class I MHC expression. For example, the K3 protein of γ-herpesvirus-68 (γ-HV68) targets ER degradation (Stevenson et al., 2000) by a mechanism that impairs heavy chain assembly with β₂m.

As a novel approach to make class I MHC molecules more stable and thus more potent stimulators of T cells and antibodies, components of the class I MHC heterotrimer have been engineered so that they are covalently attached to each other. For example, Mottez et al. (1995) reported a construct encoding a K^{d} ligand along with a linker sandwiched between the leader sequence and the N end of the mature K^{d} heavy chain. This class I MHC molecule appeared to be structurally intact and functional as assessed by T cell recognition. A serious obstacle to extending this approach to all class I MHC/peptide complexes is that the configuration of the peptide does not stably bind to the heavy chain. The widespread application of this approach is precluded by the difficulties in the expression of the class I MHC molecules, because of constraints imposed by the closed architecture of the ligand binding groove and the importance of terminal peptide residues for stable heavy chain binding (Madden et al. 1992; Matsumura et al. 1992).

Several groups have reported successfully coupling β₂m to the N terminus of different class I MHC molecules with a linker (Mage et al. 1992; Toshitani et al, 1996; Chung et al, 1999). These β₂m-heavy chain constructs maintain covalent association without altering peptide binding specificity. More recently, others have produced constructs with the peptide covalently attached to free β₂m (Uger and Barber, 1998; Uger et al. 1999; White et al., 1999). However, it remains unclear the extent to which covalently attaching peptide to β₂m excludes the binding of competing free peptide ligands. Furthermore, whether the peptide is tethered to the heavy chain or the light chain, the remaining third component may require chaperone assistance to complete the class I MHC heterotrimer.

WO 96/04314 describes “fusion complexes” of MHC molecules, molecules in which a presenting peptide is covalently bound to an MHC molecule. In some embodiments, the MHC fusion complexes can include include a flexible linker sequence interposed between the MHC molecule and the presenting peptide. (p.5, 1.. 19-21.) WO 96/04314 also refers to a single-chain fusion complex—a molecule in which the α and β chains of a Class II molecule are covalently linked to one another, in some embodiments with a linker. WO 96/04314 does not describes a single-chain fusion complex of a Class I molecule.

The component structure of MHC class I and class II molecules are very different. More specifically, the peptide binding domains, the α and β1 domains, of the class II molecule are on separate chains, whereas with class I molecules the peptide binding domains, the α1/α2 domains, are on the same chain. Furthermore, in the case of class I molecules, β₂m, does not directly contact the peptide, whereas both chains of class II molecule are required for peptide binding. Another significance difference between class I and class II molecules is that the ligand binding groove of class I molecules is closed making it highly resistant to peptide extensions (Maddem et al 1992; Matsumura et al. 1992). By contrast class II molecules clearly bind peptides that extend from the ends of its peptide binding groove.

These differences between class I and class II molecules have a clear impact on the ability to engineer an MHC molecule with covalently attached peptide. In the case of class II molecules, the peptide can be bound to the end of one of the β chain with a flexible linker and these constructs. Such constructs have been reported to efficiently fold with a chains and effectively exclude other peptides from binding (Ignatowicz et al. 2000). However, such an approach can not be used in the case of class I molecules. With class I molecules, it was not clear how to bind the peptide, because of the closed ends of the peptide binding groove. Indeed only a few cases of a peptide bound to a class I heavy chain have been reported, and in these cases it is not clear that the peptide remains covalently attached. Furthermore, binding a peptide to β₂m does not prevent other peptides from binding to the class I heavy chain.

Additionally, preassembled complexes with other proteins, such as class II MHC/peptide (Ignatowicz et al. 1996), class I MHC/class II MHC (Olson et al. 1993) and TCR/peptide (Hennecke et al. 2000) have been reported.

### SUMMARY OF THE INVENTION

Now in accordance with the invention there has been found a recombinant DNA molecule comprising a DNA sequence that encodes a novel single chain trimer (“SCT”) of a mature class I MHC molecules. The SCT contains, in sequence from the N-terminus to the C-terminus: a peptide ligand segment; (2) a first linker; (3) a β₂m segment; (4) a second linker; and (5) a class I heavy chain segment, wherein the peptide ligand segment has a carboxy end, the β₂m segment has amino and carboxy ends, and the heavy chain segment has an amino end, wherein the peptide ligand segment is covalently linked via its carboxy end to the amino end of the β₂m segment by the first linker, wherein the β₂m segment is covalently linked via its carboxy end to the amino end of the heavy chain segment by the second linker.

Representative heavy chain segments are comprised of heavy chains that include HLA-A, HLA-B, HLA-C, 1^{a}, 1^{b},H-2-K, H-2-D^{d}, and H-2-L^{d} heavy chains. In some embodiments, the heavy chain contains a mutated conserved residue. Preferably the tyrosine at position 84 in the natural sequence of the heavy chain is mutated.

The first linker preferably comprises at least 10 amino acids, more preferably at least 15 amino acids, while the second linker comprises at least 15 amino acid residues, more preferably at least 20 amino acid residues. In some embodiments, the first and second linkers contain at least about 80 percent glycine, alanine or serine residues.

In some embodiments, the peptide ligand segment is comprised of an antigenic peptide, preferably containing from about 4 to 30 amino acid residues, more preferably from about 6 to 20 amino acid residues, and still more preferably from about 8 to 12 amino acid residues. Also in accordance with the invention, there has been found a novel vector containing such SCTs is contained in a vector and a host transformed with the vector.

The inventive SCT is more resistant to down regulation by viruses and tumors, than is its non-covalently linked counterpart. Consequently, the inventive SCT is useful at eliciting T cells and antibodies to specific class I/peptide ligand complexes. This property make the SCT useful in reagents for I) making improved antibodies to enumerate class I/peptide ligand complexes in human disease, ii) making improved reagents to enumerate immune T cells in human disease, and iii) making DNA vaccines capable of eliciting specific immunity against tumors and pathogens.

### BRIEF DESCRIPTION OF THE FIGURES

FIG. 1 is a series of graphs illustrating the serologic and T cell recognition of L^{d}-derived SCTs.

FIG. 2 is a series of graphs illustrating the serologic and T cell recognition of varying OVA./β₂m^{b}.K^{b} compositions.

FIG. 3 is a graph illustrating resistance of an OVA. β₂m^{b}.K^{b} SCT to displacement by high affinity K^{b} binding peptide.

FIG. 4 is a series of graphs illustrating serologic and T cell recognition of varying OVA. β₂m^{b}.K^{b.} SCTs.

FIG. 5 is a series of graphs illustrating biochemical comparisons that include OVA. β₂m^{b}.K^{b.} SCTs.

FIG. 6(A) illustrates the superior immunogenicity of LM1.8-OVA. β₂m^{b}.K^{b} SCT (15/20) stimulators over peptide fed LM1.8-β₂m^{b} (L20).K^{b} stimulators. Lysis of RMA targets in the absence (open triangle) or continuous presence (closed triangle) of 1×10⁻⁶M SIINFEKL peptide by (C3H×B6) F1 effectors after 5 weekly stimulations with LM1.8-OVA. β₂m^{b}.etK^{b} (15/20) cells or LM1.8-β₂m^{b} (L20).K^{b} cells pulsed with continuous SIINFEKL peptide.

FIG. 6(B) illustrates that the OVA. β₂m^{b}.K^{b} (15/20) SCT construct is resistant to downregulation by the γ-HV68 encoded K3 molecule. Cell surface H-2D^{k} staining with 15-5-5 (dashed line) and H-2K^{b} staining with B8-24-3 (thick line) of LM1.8-OVA. β₂m^{b}.K^{b} (15/20) was compared both before (panel a) and after (panel b) stable expression of K3 cDNA. As a control, in panel c the endogenous K^{b} expression in B6/WT-3 cells was also monitored before (thick line) and after (dotted line) after K3 was stably introduced. The K^{b} constructs used is this figure were tagged with the 64-3-7 epitope (Myers et aL, 2000).

### DETAILED DESCRIPTION OF THE PREFERRED EMBODIMENTS

Now in accordance with the invention there has been discovered a single chain trimer (“SCT”) of a mature, single chain class I MHC molecule comprising covalently linked in sequence, beginning with the amino terminus: (1) a peptide ligand segment, (2) a flexible peptide linker, (3) a β₂m segment, (4) a flexible peptide linker, and (5) a class I heavy chain segment. These SCTs i) undergo expeditious heavy chain folding and ER to Golgi transport, ii) remain covalently attached, iii) are at least 1000 fold less accessible to exogenous peptide than class I molecules loaded with endogenous peptides, and iv) are potent simulators of peptide-specific cytotoxic T lymphocytes (“CTL”). Furthermore, these SCTs reduce or circumvent immune evasion by viruses and tumors. These molecules have application as DNA vaccines against virus infection or tumors, as well as probes of molecular mechanisms of class I assembly.

The amino acid sequences of class I heavy chains that comprise the class I heavy chain segment, as well as nucleic acids encoding these proteins, are well known in the art and are available from numerous sources including GenBank. Exemplary sequences are provided in Browning et al. (1995) (human HLA-A), Kato et al. (1993) (human HLA-B), Steinle et al. (1992) (human HLA-C), Walter et al. (1995) (rat^{a}1), Walter et al. (1994) (rat 1^{b}), Kress et al. (1983) (mouse H-2-K), Schepart et al. (1986) (mouse H-2-D^{d}), and Moore et al. (1982) (mouse H-2-L^{d}).

The present invention also provides sequence variants, also referred to as mutant proteins (muteins), of the class I heavy chain. In some embodiments, the heavy chain is modified by mutating a conserved residue, such as tyrosine at position 84 in the natural sequence, thereby causing the substation of a conservative amino acid for tyrosine. Conservative substitutions are preferred. By conservative substitution is meant replacement of an amino acid of the class I heavy chain by an amino acid which has similar characteristic and which is not likely to have an adverse effect on the heavy chain. In three dimensional structure, the tyrosine-84 residue closes the end of the binding groove preventing carboxy terminal extensions of the peptide. (Matsumura et al., Science 257:927, 1992.) Without wishing to be bound by a theory of the invention, it is believed that such a mutation opens the end of the grove where the C-end of the peptide segment sits to produce an SCT that is more stable and better recognized by T-cells and antibodies.

The novel muteins of the present invention are conventionally prepared by causing site-directed mutagenesis at the appropriate location on the gene coding for the heavy chain. Site-directed mutagenesis methods (Wallace et al., 1981, Nucleic Acids Res. 9, 3647-3656; Zoller and Smith, 1982, Nucleic Acids Res. 10, 6487-6500; and Deng and Nickoloff, 1992, Anal. Biochem. 200, 81-88) permit the replacement of tyrosine-84 with any other amino acid. Chemical synthesis of the polypeptide fragment is not beyond the scope of the present invention; however, such techniques are generally applied to the preparation of polypeptides that are relatively short in amino acid length.

The peptide linkers are flexible so as not hold the components of the SCT in undesired conformations. The linkers preferably predominantly comprise amino acids with small side chains, such as glycine, alanine and serine, to provide for flexibility. Preferably at least about 80 percent of the linkers comprise glycine, alanine or serine residues, particularly glycine and serine residues. Preferably, the linkers do not contain any proline residues, which could inhibit flexibility. Different linkers can be used including any of a number of flexible linker designs that have been used successfully to join antibody variable regions together (see M. Whitlow et al., Methods: A Companion to Methods in Enzymology, 2:97-105 (1991). Suitable linkers can be readily identified empirically. For example, a DNA construct coding for an SCT that includes the linker can be cloned and expressed, and the molecule tested to determine if it is capable of modulating the activity of a T cell receptor, either to induce T-cell proliferation or to inhibit or inactivate T cell development. Suitable size and sequences of linkers also can be determined by conventional computer modeling techniques based on the predicted size and shape of the SCT.

A linker is interposed between the heavy chain segment and the β₂m segment. For covalently linking the heavy chain and the β₂ m, the linker spans from the N-end of the heavy chain segment to the C-end of the β₂m segment. When such a heavy chain/β₂m is expressed, the heavy chain and the β₂m should fold into the binding groove resulting in a functional. Preferably the first linker comprises at least 10 amino acids, more preferably at least 15 amino acids. Without wishing to be bound by a theory of the invention, it is believed that the first flexible linker allows the β₂m to properly align itself with the heavy chain so as to become effectively associated with the heavy chain and form a binding groove, while minimizing or eliminating dissociative effects that might otherwise be imparted by viruses or tumors.

The β₂m used to form the β₂m segment can be obtained from a variety of sources, including, for example, human, murine, bovine, equine or other mammalian serum or body fluids normally containing a small amount of free β₂m. Mixtures of β₂m from these sources can also be used. Purified human β₂m is available commercially, for example from Sigma Chemical Co., St. Louis, Mo. Alternatively, β₂m can be isolated and purified from serum or other body fluids using conventional techniques or can be produced by recombinant techniques based upon the introduction of β₂m genes into appropriate expression systems. The human and murine genes encoding β₂m have previously been cloned. In addition, their sequences are known, thus allowing for the isolation of a DNA clone from these or other species.

Another flexible linker is interposed between the β₂m segment and the peptide ligand segment. For covalently linking the β₂m segment and the peptide ligand segment the linker spans from the N-end of the β₂m segment to the C-end of the peptide ligand segment. When such a β₂m/peptide ligand chain is expressed along with the heavy chain, the linked peptide ligand should fold into the binding groove resulting in a functional SCT. Preferably, this linker comprises at least 15 amino acids, more preferably about 20 amino acids. Without wishing to be bound by a theory of the invention, it is believed that this flexible linker allows effective positioning of the peptide ligand with respect to the binding groove, while minimizing or eliminating dissociative effects that might otherwise be imparted by viruses or tumors.

The term peptide is used interchangeably with polypeptide to designate a series of amino acids connected one to the other by peptide bonds between the alpha-amino and alpha-carboxy groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy their biological activity.

As used herein, the term “peptide ligand” refers to a peptide, glycopeptide, glycolipid or any other compound associated the ligand binding groove of various different molecules with an MHC class I or MHC class I-like structure (Fundamental Immunology, 2d Ed., W. E. Paul, ed., Ravens Press N.Y. 1989). Preferred peptides include peptides that are capable of modulating the activity of a T cell receptor, either to induce T-cell proliferation, to inhibit or inactivate T cell. Antigenic peptides from a number of sources have been characterized in detail, including antigenic peptides from honey bee venom allergens, dust mite allergens, toxins produced by bacteria (such as tetanus toxin) and human tissue antigens involved in autoimmune diseases. Detailed discussions of such peptides are presented in U.S. Pat. Nos. 5,595,881, 5,468,481 and 5,284,935. Exemplary peptides include those identified in the pathogenesis of rheumatoid arthritis (type II collagen), myasthenia gravis (acetyl choline receptor), and multiple sclerosis (myelin basic protein). As an additional example, suitable peptides which induce Class I MHC-restricted CTL responses against HBV antigen are disclosed in U.S. Pat. No. 6,322,789.

As is well known in the art (see, for example, U.S. Pat. No. 5,468,481) the presentation of antigen in MHC complexes on the surface of APCs generally does not involve a whole antigenic peptide. Rather, a peptide located in the groove is typically a small fragment of the whole antigenic peptide. As discussed in Janeway & Travers (1997), peptides located in the peptide groove of Class I MHC molecules are constrained by the size of the binding pocket and are typically 8-15 amino acids long, more typically 8-10 amino acids in length (but see Collins et al., 1994 for possible exceptions).

In addition to antigenic peptides, the peptide ligands can also comprise autologous, or “self” peptides. If T lymphocytes then respond to cells presenting “self” peptides, a condition of autoimmunity results. See, Buus, S., et al., Science 242:1045-1047 (1988); Demotz, et al., Nature 342:682-684 (1989). Over 30 autoimmune diseases are presently known, including myasthenia gravis (MG), multiple sclerosis (MS), systemic lupus erythematosis (SLE), rheumatoid arthritis (RA), insulin-dependent diabetes mellitus (IDDM), etc. Characteristic of these diseases is an attack by the immune system on the tissues of the victim. In nondiseased individuals, such attack does not occur because the immune system is tolerant of “self”, i.e., it does not recognize “self” tissues as foreign; however, in persons suffering from autoimmune diseases, such tolerance does not occur and tissue components are recognized as foreign. For a general review of autoimmune disease, see, Sinha et al., Science 248:1380-1387 (1990).

The peptide ligand generally will be as small as possible while still maintaining substantially all of the biological activity of the large peptide. Preferably, the peptide ligand has from about 4 to 30 amino acid residues, more preferably about 6 to about 20 amino acid residues. When possible, it may be desirable to optimize the peptide ligands to the preferred length of 8 to 12 amino acid residues, commensurate in size with endogenously processed viral peptides that are bound to Class I MHC molecules on the cell surface. See generally, Schumacher et al., Nature 350:703-706 (1991); Van Bleek et al., Nature 348:213-216 (1990); Rotzschke et al., Nature 348:252-254 (1990); and Falk et al., Nature 351:290-296 (1991). The activity of a particular peptide ligands, i.e., antigenic or antagonist or partial agonist, can be readily determined empirically by methods well known in the art, including by in vivo assays.

In general, preparation of the inventive SCTs can be accomplished by procedures disclosed herein and by recognized recombinant DNA techniques, e.g., preparation of plasmid DNA, cleavage of DNA with restriction enzymes, ligation of DNA, transformation or transfection of a host, culturing of the host, and isolation and purification of the expressed fusion complex. Such procedures are generally known and disclosed e.g in Sambrook et al., Molecular Cloning (2d ed. 1989).

More specifically, DNA coding for a desired class I heavy chain is obtained from a suitable cell line. Other sources of DNA coding for the class I heavy chain are known, e.g., human lymphoblastoid cells. Once isolated, the gene coding for the class I heavy chain can be amplified by the polymerase chain reaction (PCR) or other means known in the art. The PCR product also preferably includes a sequence coding for the linkers, or a restriction enzyme site for ligation of such a sequence.

To make a vector coding for an SCT, the sequence coding for the heavy chain and the β₂m is linked to a sequence coding for the peptide ligand by use of suitable ligases. DNA coding for the peptide ligand can be obtained by isolating DNA from natural sources or by known synthetic methods, e.g., the phosphate triester method. See, e.g., Oligonucleotide Synthesis, IRL Press (M. Gait, ed., 1984). Synthetic oligonucleotides also may be prepared using commercially available automated oligonucleotide synthesizers. A DNA sequence coding for the linkers as discussed above is interposed between the sequence coding for the β₂m segment and the sequence coding for the peptide ligand segment and between the β₂m segment and the heavy chain segment and the segments are joined using suitable ligases.

Other nucleotide sequences also can be included in the gene construct. For example, a promoter sequence, which controls expression of the sequence coding for the β₂m segment covalently bound to the peptide ligand segment, or a leader sequence, which directs the heavy chain segment to the cell surface or the culture medium, can be included in the construct or present in the expression vector into which the construct is inserted. An immunoglobulin or CMV promoter is particularly preferred. A strong translation initiation sequence also can be included in the construct to enhance efficiency of translational initiation. A preferred initiation sequence is the Kozak consensus sequence (CCACCATG).

Preferably, a leader sequence included in a DNA construct contains an effectively positioned restriction site so that an oligonucleotide encoding a peptide ligand segment of interest can be attached to the first linker. Suitably the restriction site can be incorporated into the 3-end of the leader sequence, sometimes referred to herein as a junction sequence, e.g., of about 2 to 10 codons in length, that is positioned before the coding region for the peptide ligand. A particularly preferred restriction site is the AflII site, although other cleavage sites also can be incorporated before the peptide ligand coding region. As discussed above, use of such a restriction site in combination with a second restriction site, typically positioned at the beginning of the sequence coding for the linker, enables rapid and straightforward insertion of sequences coding for a wide variety of peptide ligands into the DNA construct for the SCT. Preferred leader sequences contain a strong translation initiation site and a cap site at the 3′-end of their Mrna. Preferably a leader sequence is attached to the heavy chain. Preferred leader sequences provides for secretory expression of the SCT.

A number of strategies can be employed to express SCTs of the invention. For example, the SCT can be incorporated into a suitable vector by known means such as by use of restriction enzymes to make cuts in the vector for insertion of the construct followed by ligation. The vector containing the SCT is then introduced into a suitable host for expression. See, generally, Sambrook et al., supra. Selection of suitable vectors can be made empirically based on factors relating to the cloning protocol. For example, the vector should be compatible with, and have the proper replicon for the host that is being employed. Further the vector must be able to accommodate the DNA sequence coding for the SCT that is to be expressed. Suitable host cells include eukaryotic and prokaryotic cells, preferably those cells that can be easily transformed and exhibit rapid growth in culture medium. Specifically preferred hosts cells include prokaryotes such as E. coli, Bacillus subtillus, etc. and eukaryotes such as animal cells and yeast strains, e.g., S. cerevisiae. Mammalian cells are generally preferred, particularly J558, NSO, SP2-O or CHO. Other suitable hosts include, e.g., insect cells such as Sf9. Conventional culturing conditions are employed. See Sambrook, et al., supra. Stable transformed or transfected cell lines can then be selected. Cells expressing an SCT can be determined by known procedures. For example, expression of an SCT linked to an immunoglobulin can be determined by an ELISA specific for the linked immunoglobulin and/or by immunoblotting.

An expressed SCT can be isolated and purified by known methods. Typically, the culture medium is centrifuged and then the supernatant is purified by affinity or immunoaffinity chromatography, e.g., Protein-A or Protein-G affinity chromatography or an immunoaffinity protocol comprising use of monoclonal antibodies that bind the expressed fusion complex such as a linked MHC or immunoglobulin region thereof. For example, SCTs containing human HLA-DR1 sequences can be purified by affinity chromatography on a monoclonal antibody L243-Sepharose column by procedures that are generally known and disclosed, e.g., see Harlow, E. et al., Antibodies, A Laboratory Manual (1988). The L243 monoclonal antibody is specific to a conformational epitope of the properly folded HLA-DR1 molecule (J. Gorga et al., J. Biol. Chem., 262:16087-16094), and therefore would be preferred for purifying the biologically active SCT. The SCT also may contain a sequence to aid in purification; e.g., a 6×His tag.

The SCTs in accordance with the invention are useful in mediating cell immunity as evidenced by their ability to generate a cytotoxic T lymphocytes specific for class I/peptide complexes. Furthermore, plasmid DNA that encodes the inventive SCT may induce the expression of specific antibodies, a response known to be dependent upon helper T cells.

The SCTs of the invention and compositions containing antigens bound to the SCTs are useful for the preparation of antibodies that recognize these substances. The antibodies have diagnostic uses, application in mammalian therapy, and use in the study of MHC and cellular processes.

More particularly, polyclonal or monoclonal antibodies can be used in a variety of applications. Among these the neutralization of MHC gene products by binding to the gene products on cell surfaces. They can also be used to detect MHC gene products in biological preparations or in purifying corresponding MHC gene products or SCTs of the invention, such as by affinity chromatography.

Antibodies according to the present invention can be prepared by any of a variety of methods. For example, cells expressing the SCT or a functional derivative thereof can be administered to an animal in order to induce the production of sera containing polyclonal antibodies that are capable of binding the SCT In addition, antibodies can be prepared to the SCTs of the invention and compositions containing antigens bound to the molecules in a similar manner.

In a preferred method, the antibodies are monoclonal antibodies, which can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., In: Monoclonal Antibodies and T-Cell Hybridomas, Elsevier, N.Y., pp. 563-681 (1981)). In general, such procedures involve immunizing an animal with the SCT or the SCT-antigen composition. Splenocytes of the animals are extracted and fused with a myeloma cell line. After fusion, the resulting hybridoma cells can be selectively maintained in HAT medium, and then cloned by limiting dilution as described by Wands, J. R., et al. Gastroenterology 80:225-232 (1981). The hybridoma cells obtained are then assayed to identify clones secreting antibodies capable of binding the SCT or the composition.

See also U.S. Pat. No. 2,658,197 (A1) [90 01769], Feb. 14, 1990, “Restricted Monoclonal Antibodies That Recognize A Peptide That Is Associated With An Antigen Of A Major Histocompatibility Complex, Use In Diagnosis and Treatment, “Huynh Thien Duc Guy, Pririe Rucay, Philippe Kourilsky; National Institute of Health and Medical Research.

The antibodies can be detectably labeled. Examples of labels that can be employed in the present invention include, but are not limited to, enzymes, radioisotopes, fluorescent compounds, chemiluminescent compounds, bioluminescent compounds, and metal chelates.

Examples of enzymes include malate dehydrogenase, staphylococcal nuclease, delta-V-steroid isomerase, yeast alcohol dehydrogenase, alpha-glycerophosphate dehydrogenase, triose phosphate isomerase, biotin-avidin peroxidase, horseradish peroxidase, alkaline phosphatase, asparaginase, glucose oxidase, β.-galactosidase, ribonuclease, urease, catalase, glucose-VI-phosphate dehydrogenase, glucoamylase and acetylcholine esterase.

Examples of isotopes are ³H, ¹²⁵I, ³²P, ³⁵S, ¹⁴C, ⁵¹Cr, ³⁶Cl, ⁵⁷Co, ⁵⁸Co, ⁵⁹Fe, and ⁷⁵Se. Among the most commonly used fluorescent labeling compounds are fluoroscein, isothiocyanate, rhodamine, phycoerythrin, phycocyanin, allophycocyanin, o-phthaldehyde, and fluorescamine. Examples of typical chemiluminescent labeling compounds are luminal, isoluminol, theromatic acridinium ester, imidazole, acridinium salts, oxalate ester, and dioxetane.

Those of ordinary skill in the art will know of other suitable labels for binding to antibodies, or will be able to ascertain the same by the use of routine experimentation. Furthermore, the binding of these labels to antibodies can be accomplished using standard techniques commonly known to those of ordinary skill in the art. Bioluminescent compounds for purposes of labeling include luciferin, luciferase and aequorin.

The antibodies and antigen of the present invention are ideally suited for the preparation of a kit. Such kit may comprise a carrier means being compartmentalized to receive one or more container means, such as vials, tubes and the like, each of said container means comprising the separate elements of the assay to be used.

The SCTs, compositions containing antigens bound to the SCTs, and antibodies to these substances are useful in diagnostic applications. For example, the SCTs can be used to target lymphocyte receptors, such as CD4⁺ and CD8⁺ receptors of T lymphocytes, and the resulting bound determinant can be assayed, for instance, by means of an antibody to the bound determinant. In addition, it will be understood that the SCTs of the invention can be labeled in the manner previously described for antibodies. In this case, the label on the molecule can be detected and quantified. Compositions comprising an antigen bound to an SCTs of the invention can be used in a similar manner with MHC-restricted receptors recognizing the antigen and the determinant. Typical examples of assays based on the antibodies of the invention are radioimmunoassays (RIA), enzyme immunoassays (EIA), enzyme-linked immunosorbent assays (ELISA), and immunometric or sandwich immunoassays, including simultaneous sandwich, forward sandwich, and reverse sandwich immunoassays.

In the preferred mode for performing the assays, it is desirable to employ blockers in the incubation medium to assure that non-specific proteins, protease or human antibodies to immunoglobulins present in the experimental sample do not cross-link or destroy the antibodies and yield false positive or false negative results. Nonrelevant (i e., nonspecific) antibodies of the same class or subclass (isotype) as those used in the assays (e.g., IgG, IyM, etc.) can be used as blockers. In addition, a buffer system should be employed. Preferred buffers are those based on weak organic acids, such as imidazole, HEPPS, MOPS, TES, ADA, ACES, HEPES, PIPES, TRIS, and the like, at physiological pH ranges. Somewhat less preferred buffers are inorganic buffers such as phosphate, borate or carbonate. Finally, known protease inhibitors can be added to the buffer.

Well known solid phase immunoadsorbents, such as glass, polystyrene, polypropylene, dextran, nylon and other materials, in the form of tubes, beads, and microtiter plates formed from or coated with such materials, can be employed in the present invention. Immobilized antibodies can be either covalently or physically bound to the solid phase immunoadsorbent by techniques such as covalent bonding via an amide or ester linkage, or by adsorption.

In another embodiment of this invention, the SCTs and compositions containing antigens bound to the SCTs and antibodies to these substances can be administered to a mammal to produce a therapeutic effect. For example, immune responses to self components represent a failure of immunological tolerance. As a result, clones of T cells and B cells emerge bearing receptors for self-antigens, which can lead to the production of self-directed antibodies, cytotoxic T cells, and inflammatory T cells. Such a breakdown in tolerance produces an autoimmune response that can cause autoimmune diseases. Administration of the SCTs, compositions, or antibodies of the invention can intervene in these processes. Thus, for example, this invention can be utilized to treat T cell mediated autoimmune diseases, such as thyroiditis and multiple sclerosis. Other therapeutic uses include therapeutics for bacterial and viral infections, as well as for cancer treatments.

This invention also provides SCTs for use in therapeutic or vaccine compositions. Conventional modes of administration can be employed. For example, administration can be carried out by oral, respiratory, or parenteral routes. Intradermal, subcutaneous, and intramuscular routes of administration are preferred when the vaccine is administered parenterally.

The ability of the SCTs of the invention to exhibit a therapeutic or immunizing effect can be enhanced by emulsification with an adjuvant, incorporation in a liposome, coupling to a suitable carrier or even in cells or by combinations of these techniques. For example, the molecules and compositions can be administered with a conventional adjuvant, such as aluminum phosphate and aluminum hydroxide gel, in an amount sufficient to mediate humoral or cellular immune response in the host. Other suitable water soluble adjuvants, such as the Ribi adjuvant system available from Corixa, Seattle, Wash.

Similarly, these reagents can be bound to lipid membranes or incorporated in lipid membranes to form liposomes. The use of nonpyrogenic lipids free of nucleic acids and other extraneous matter can be employed for this purpose.

In addition, any of the common liquid or solid vehicles can be employed, which are acceptable to the host and do not have any adverse side effects on the host nor any detrimental effects on the reagents of the invention. Conveniently, phosphate buffered saline at a physiological PH can be employed as the carrier. One or more injections may be required, particularly one or two additional booster injections. It will be understood that conventional adjuvants, such as SAF-1, complete Freund's adjuvant and incomplete Freund's adjuvant, or oil-based adjuvants, such as mineral oil, can be administered with the reagents of the invention to elicit an increased antibody or cell-mediated immune response.

The immunization schedule will depend upon several factors, such as the susceptibility of the host and the age of the host. A single dose of the reagents of the invention can be administered to the host or a primary course of immunization can be followed in which several doses at intervals of time are administered. Subsequent doses used as boosters can be administered as needed following the primary course.

In addition to the antibodies produced for kits and diagnostic assays, antibodies of the present invention can be humanized by procedures well known in the art (using either chimeric antibody production or CDR grafting technology). U.S. Pat. No. 4,816,567 Cabilly et al., EPA 0120694 Publication No., assigned to Celltech, EPA 0173494 Publication No. assigned to Stanford University, and EPA 0125023 Publication No. assigned to Genentech, describing chimeric antibody procedures and EPA 0194276 Publication No. assigned to Celltech describing CDR grafting procedures.

The humanized antibodies would be prepared from antibodies obtained against specific MHC-antigen complexes. The humanized antibodies could then be used therapeutically in humans so as to avoid the problems associated with the use of non-human antibodies in human therapy.

This invention will now be described in greater detail in the following Examples.

### EXAMPLES

Single chain trimers of Class I MHC molecules, where all three components of the completely assembled class I molecules are covalently attached to each other via flexible peptide linkers were produced. Each of the SCTs consisted of the following elements beginning with the amino terminus: a leader sequence of μ₂m, the peptide encoding a ligand for the heavy chain, a first flexible linker of 10 or 15 amino acid residues, the mature portion of murine β₂m, a second flexible linker of 15 or 20 amino acid residues, and finally the mature portion of a heavy chain.

To serve as controls, constructs were also made with only β₂m covalently attached to a heavy chain. The control constructs consisted of the entire coding region of β₂m^{b} linked via a 15 or 20 amino acid residue linker to the mature portion of the respective heavy chain.

These constructs were stably introduced into mouse or human cell lines and cloned by limiting dilution. Structural integrity of these constructs was then examined by serological as well as functional assays.

Mice

B6 (H-2^{b}), BALB/c (H-2^{d}) and (C3H×B6)F1 (H-2^{kxb}) were purchased from Charles River Laboratory (Wilmington, Mass.) and housed in the barrier animal facility at Washington University School of Medicine (St. Louis, Mo.). OT-1 transgenic mice (Hogquist et al., 1994) were obtained from the Washington University School of Medicine.

Cell Lines, Antibodies and Peptides

Cell lines used in this study were RMA, LM1.8, DLD-1, and B6/WT-3. RMA is a Rauscher leukemia virus-induced cell line of C57BL/6 (H-2^{b}) origin. LM1.8 was obtained from INSERM, Institut Pasteur, France and was derived by introducing the mouse ICAM-1 Cdna into the mouse Ltk⁻ fibroblast line DAP-3 under HAT selection (Jaulin et al., 1992). DLD-1 cells which were derived from human colon carcinomas (Dexter et al., 1979) were purchased from ATCC (Rockville, Md.). The B6/WT-3 cells were derived by SV40 transformation of C57BL/6 embryo fibroblasts as described by Pretell et al. (1979) and were obtained from Louisiana State University Health Sciences Center, Shreveport, La.

MAbs used in this study included the followings: 30-5-7 and 64-3-7 which recognize the folded and open forms of L^{d}, respectively (Lie et al., 1991 and Smith et al., 1992); mAbs B8-24-3 and 15-5-5 (purchased from ATCC) which recognize folded K^{b} and D^{k}, respectively; mAb 25D-1.16 (obtained from, NIH, Rockville, Md.) which recognizes K^{b}+SIINFEKL peptide (Porgador et al., 1997). All cells were maintained in complete medium (either DMEM or RPMI 1640) which included 1 Mm sodium pyruvate, 0.1 Mm non-essential amino acids, 2 Mm glutamine, 25 μM HEPES, and 100 U/ml penicillin/streptomycin and supplemented with 10% heat inactivated bovine calf serum (HyClone Laboratories, Logan, Utah).

The QL9 peptide (QLSPFPFDL), the OVA-derived peptide (SIINFEKL) and SIYR peptide (SIYRYYGL) were synthesized using Merrifield's solid phase method (1963) on a peptide synthesizer (model 432A: Applied Biosystems, Foster City, Calif.). Peptides were purified by reverse phase HPLC and purity (>95%) was assessed as described by Gorka et al. (1989).

DNA Constructs

Table I lists all the single chain constructs and the sequences of the covalent peptides ligands and flexible peptide linkers. All PCRs were performed using Expandase (Roche Molecular Biochemicals, Indianapolis, Ind.) under standard conditions and the amplified portions of each construct were sequenced for verification.

The β₂m^{b}.L^{d} and β₂m.K^{b} constructs were made in two steps. First, an XbaI/BamHI cut PCR fragment encoding the β₂m^{b} coding sequence and the first 10 amino acid residues of the linker were cloned into the XbaI/BamHI sites of the mammalian expression vector RSV5.neo (Long et al., 1991) to create RSV.5.neo. β₂M^{b}+linker. Second, a BamHI cut PCR fragment encoding the last 7 amino acid residues of the linker and the mature portion of either L^{d} or K^{b} Cdna were cloned into the BamHI site of RSV.5.neo. β₂m^{b}+linker to create RSV.5.neo.β₂m^{b}.L^{d}/K^{b}.

The QL9. β₂m^{b}.L^{d} construct was made by engineering an AvrII site at the junction between the QL9 peptide and the beginning of the linker. Two PCR fragments, one encoding the β₂m signal peptide and the QL9 peptide and cut with XbaI/AvrII and the other one encoding the linker +β₂m residues 1-27 and cut with AvrII/SnaBI cells were cloned into the XbaI and SnaBI sites of RSV.5.neo. β₂m.L^{d} by 3-piece ligation with the Rapid DNA Ligation Kit (Roche Molecular Biochemicals), to create RSV.5.neo.QL9. β₂m^{b}.L^{d}. To increase expression efficiency after stable transfection, all these constructs were subcloned into the Pires.neo vector (Clontech, Palo Alto, Calif.).

The MCMV. B₂m^{b}L^{d}, p29. B₂m^{b}.L^{b} and OVA. B₂m^{b}.K^{b} constructs were prepared using the same method. The epitope tagged K^{b} mutant (K^{b}R48Q, R50P) was described previously (Myers et al., 2000). The different linker variants were made by PCRs using NheI and BspEI sites engineered into the first and second linkers, respectively. The K3 Cdna was amplified by PCR from a K3 encoding plasmid kindly obtained from Washington University, St. Louis, Mo. and cloned into the EcoRI and BamHI sites of Pires.puro2 (Clontech). The various constructs were transfected into LM1.8, DLD-1 or B6/WT-3 cells using LipoFectin (Life Technologies, Gaithersburg, Md.) or Fugene 6 (Roche Molecular Biochemicals) according to manufacturer's instructions. Neomycin resistance was selected in 0.6 mg/ml geneticin (Life Technologies) and puromycin resistance was selected in 5 μg/ml puromycin (Sigma, St. Louis, Mo.).

CTL Generation and Maintenance

The L^{d}-alloreactive CTL clone, 2C, was obtained from MIT, Cambridge, Mass. It was grown in sensitzation medium [complete RPMI 1640 supplemented with 10% heat inactivated fetal calf serum (HyClone Laboratories), 50 μM 2-ME, 10U/ml Ril-2] and maintained by weekly restimulation with irradiated (2,000R) BALB/c splenocytes (2.5×10⁵ responders and 5×10⁶ stimulators) in 24 well plates at 2 ml per well. The OT-1 T cells were derived by stimulating 2.5×10⁶ OT-1 splenocytes with 5×10⁶ irradiated B6 splenocytes in sensitization medium in the presence of 5×10⁻⁶M SIINFEKL but without Ril-2 for 5 days. Thereafter, the OT-1 line was restimulated weekly with 10U/ml Ril-2 at 5×10⁵ responders per 5×10⁶ stimulators. To test the immunogenicity of the single chain constructs, 7.5×10⁶ responding (C3H×B6) F1 splenocytes were co-cultured with 3.5×10⁵ irradiated (10,000R) LM1.8-β₂m(L20).etK^{b} cells in the presence of 1×10⁻⁴M SIINFEKL peptide or LM1.8-OVA. β₂m^{b}.etK^{b} (15/20) cells in 24-well Linbro trays containing 2 ml sensitization medium without Ril-2. After 5 days, they were restimulated in sensitization medium without IL-2 at 2.5×10⁶ responders per 3.5×10⁵ stimulators with 1×10⁻⁴M SIINFEKL peptide (for LM1.8-β₂m(L20).etK^{b} cells). Thereafter, they were restimulated weekly in the presence of 10U/ml Ril-2 at 2.5-5×10⁵ responders per 3.5×10⁵ stimulators with 1×10⁻⁵M SIINFEKL peptide (for LM1.8-β₂m(L20).etK^{b} cells).

⁵¹Cr Release Assay

1×10⁶ target cells were labeled with 150-200 μCi of ⁵¹Cr (Na⁵¹CrO₄, NEN, Boston, Mass.) in 0.2-0.3 ml of complete RPMI 1640 medium +10% bovine calf serum at 37° C. in 5% CO₂ for 1-2 hours. Effector cells were plated into round bottom 96-well microtiter plates at various concentrations in the absence or continuous presence of peptide, and 2×10³ washed target cells per well were added. The plates were centrifuged at 50×g for 1 minute and incubated for 4 hours at 37° C. in 5% CO₂. Radioactivity in 100 μl of supernatant was measured in an Isomedic gamma counter (ICN Biomedicals, Huntsville, Ala.). The mean of triplicate samples was calculated, and percentage ⁵¹Cr release was determined according to the following equation: percentage ⁵¹Cr release=100×((experimental ⁵¹Cr release−control ⁵¹Cr release)/(maximum ⁵¹Cr release−control ⁵¹Cr release)), where experimental ⁵¹Cr release represents counts from target cells mixed with effector cells; control ⁵¹Cr release represents counts from target cells incubated with medium alone (spontaneous release); and maximum ⁵¹Cr release represents counts from target cells lysed by the addition of 5% Triton X-100. Spontaneous release ranged from 5-20%.

Flow Cytometry and Peptide Induction

3-5×10⁵ cells were washed and incubated on ice in FACS medium (PBS containing 1% BSA and 0.1% NaN₃) in the presence of a saturating concentration of mAb for 30-60 minutes, washed twice in FACS medium, and incubated on ice with a saturating concentration of FITC-labeled, Fc-specific goat anti mouse-IgG F(ab′)₂ (ICN Biomedicals, Aurora, Ohio) or PE-labeled, goat anti mouse IgG (Pharmingen, San Diego, Calif.) for 20 min. Cells were washed twice and resuspended in FACS medium. Viable cells, gated by forward and side scatter, were analyzed and a FACSCalibur (Becton Dickinson, San Jose, Calif.) equipped with an argon ion laser tuned to 488 nm and operating at 150Mw. The data are expressed as linear fluorescence values obtained from log-amplified data using CELLQuest Software (Becton Dickinson). Cells incubated with an irrelevant primary mAb followed by secondary antibodies were used as negative controls. For peptide incubation, 1×10⁶ cells were incubated with the indicated concentration of peptide in a final volume of 2 ml complete medium at 37° C. overnight in a 6 well plate.

Immunoprecipitation and Western Blotting.

Immunoprecipitations and Western blots. Cells were lysed in 10 Mm Tris buffered saline PH 7.4 (TBS) containing 1% digitonin (Wako, Richmond, Va.) with 20 Mm iodoacetamide (IAA) and 0.2 Mm of freshly added PSMF (Sigma). Saturating amounts of the primary antibody were added to the lysis buffer. Post-nuclear lysates were added to protein A-Sepharose CL-4B (Amersham Pharmacia, Uppsala Sweden) for 60 minutes on ice and protein A-bound material was washed in 0.1% digitonin in TBS. Immunoprecipitates were eluted from protein A by boiling for 5 minutes in elution buffer (LDS sample buffer; Invitrogen, Carlsbad, Calif.). Samples were electrophoresed on 7% tris-acetate polyacrylamide gels (Invitrogen) and transferred to Immobilon-P PVDF membranes (Millipore, Bedford, Mass.). After overnight blocking in 10% dried milk in PBS-0.05% Tween 20, membranes were incubated with mAb 64-3-7 for 1 hour, washed three times with PBS-0.05% Tween 20, and incubated for 1 hour with biotin-conjugated goat anti-mouse IgG_{2b} (Caltag, San Francisco, Calif.). Following three washes with PBS-0.05% Tween 20, membranes were incubated for 1 hour with streptavidin-conjugated HRP (Zymed, San Francisco, Calif.), washed three times with PBS-0.03% Tween 20, and incubated with ECL chemiluminescent reagents (Amersham Pharmacia Biotech, Piscataway, N.J.) prior to exposure to BioMax-MR film (Eastman Kodak, Rochester, N.Y.).

Pulse-chase and immunoprecipitations. After a 45 min pre-incubation in Met/Cys-free medium (DMEM with 5% dialyzed FCS), cells (at 20×10⁶ cells/ml) were pulse labeled with Express ³⁵S-Met/Cys labeling mix (Perkin Elmer Life Sciences, Boston, Mass.) at 300 μCi/ml for 10 min. Cells were then washed extensively, an aliquot removed for the zero time point, and the remaining cells returned to culture at 37 degrees for the indicated times. For immunoprecipitations, labeled cells were lysed in 1% NP-40 (Sigma) dissolved in TBS with 20 Mm IAA and 5 Mm PMSF. Post-nuclear lysates were pre-cleared over protein A-sepharose CL-4B for 30 min on ice. Lysates were then transferred to protein A-Sepharose pellets containing the appropriate pre-bound mAbs. After binding for 45 min on ice, protein A pellets were washed 4 times with 0.1% NP-40 in TBS, and bound proteins were eluted by boiling in 10 Mm tris-Cl, PH 6.8+0.5% SDS+1% 2-mercaptoethanol. Eluates were mixed with an equal volume of 100 Mm sodium acetate, PH 5.4 and digested overnight with 1 Mu endoglycosidase H (ICN, Costa Mesa, Calif.) that was reconstituted in 50 Mm sodium acetate, PH 5.4. Following SDS-PAGE, gels were treated with Amplify (Amersham), dried, and exposed to BioMax-MR film.

### Example 1

Correlation of the level and quality of surface expression of SCT molecules with the known affinity of peptide binding to class I when non-covalently attached. To serologically determine the quality of the SCT and test the role of peptide affinity, an SCT was prepared containing an L^{d} heavy chain and a QL9 peptide ligand, along with β₂m.

The L^{d} heavy chain has well characterized mAbs that distinguish L^{d} heavy chain conformation as determined by occupancy with high affinity peptide ligands (Lie et al. 1991; Smith et al., 1992 and 1993; Yu et al., 1999). More specifically, two mAbs, 30-5-7 and 64-37 recognize the folded (peptide loaded) and open (peptide empty) conformers of L^{d}. Evidence for the reciprocal specificity of the two mAbs includes the fact that incubation with high affinity peptide ligands leads to an increase in 30-5-7⁺L^{d} and a decrease in 64-3-7⁺L^{d}, whereas acid stripping leads to a sharp decrease in 30-5-7⁺L^{d} and a proportional increase in 64-3-7⁺L^{d}. Thus these two mAbs can be used in tandem to assess the effect of covalent linkage on the expression of the resultant SCT.

For the ligand, sequence encoding the nonomeric peptide termed QL9 (Sykulev et al. 1994) was initially used to make the single chain construct QL9. B₂m^{b}.L^{d}. The QL9 peptide is recognized by a well characterized Ld-restricted alloreactive CTL clone 2C (Udaka et al., 1992). As a peptide minus control construct, β₂m^{b}.L^{d}, was generated by linking β₂m and L^{d} together with a 15 residue flexible linker. These two constructs, QL9. B₂m^{b}.L^{d} and β₂m^{b}.L^{d}, were then stably transfected into the human cell line DLD-1, which fails to express endogenous β₂m (Bicknell et al., 1994). Clonal transfectants expressing QL9. B₂m^{b}.L^{d} or β₂m^{b}.L^{d} were then examined by flow cytometry with mAbs 30-5-7 and 64-3-7.

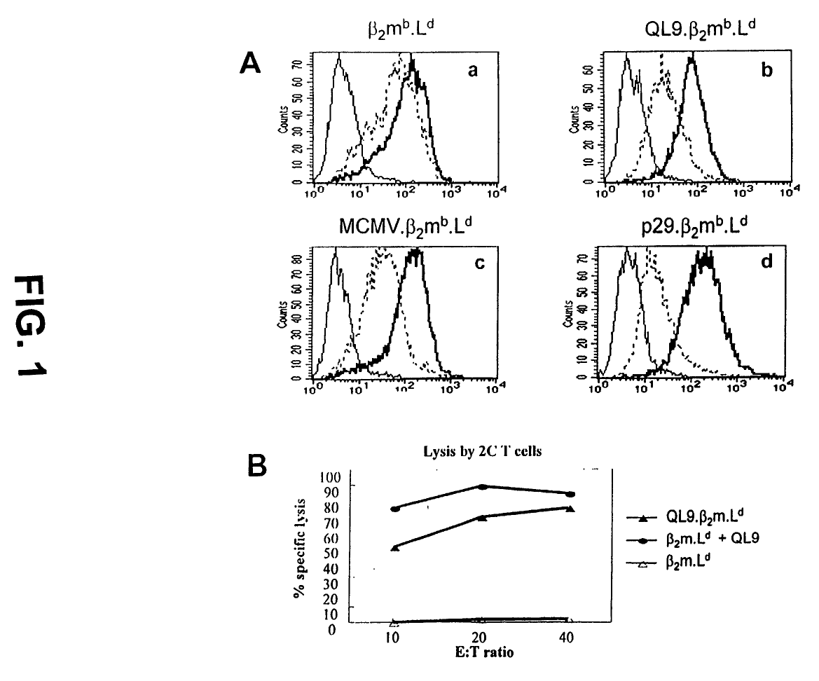

As shown in FIG. 1A (parts a and b), both constructs were expressed on the surface of the DLD-1 transfectants indicating that covalent linkage of β₂m can override the requirement for endogenous, β₂m, in agreement with published observations (Lee et al. 1994; Toshitani et al., 1996). In addition, it was found that the QL9. B₂m^{b}.L^{d} construct containing all three elements of fully assembled L^{d} can fold correctly and be expressed on the cell surface as detected by the mAb 30-5-7 that detects an L^{d} conformation acquired after binding high affinity peptide ligands.

A comparison of the percentage of QL9. B₂m^{b}.L^{d} vs. β₂m^{b}.L^{d} in the open vs. folded conformation was also made. Whereas 39% of surface β₂m^{b}.L^{d} molecules were detected in an open conformation, only 22% of surface QL9. B₂m^{b}.L^{d} were detected in an open conformation. This difference suggests that covalent attachment of peptide improved the efficiency of peptide loading and reduced, but did not eliminate peptide dissociation. Relative to other class I molecules the L^{d} molecule is known to be highly susceptible to peptide and β₂m dissociation (Hansen et al., 2000). Indeed this propensity to disassemble results in normal (unattached) L^{d} having a lower level of surface expression relative to other class I molecules. The propensity to disassemble makes L^{d} an ideal candidate to test the role of peptide affinity in expression of SCT molecules. For these comparisons, SCT molecules were constructed that included two different L^{d} ligands, MCMV (Reddehase et al. 1989) and p29 (Corr et al., 1992). In previously published data, it was determined that QL9/L^{d} and MCMV/L^{d} complexes have a half life of about 2 hours, whereas p29/L^{d} complexes have a half live of greater that 6 hours (Smith et al., 1992). Indeed the p29 peptide was the only peptide to fold recombinant L^{d} heavy chains to a sufficient extent to obtain crystals (Balendiran et al., 1997). In agreement with the studies using L^{d} ligands in solution, the MCMV. B₂m^{b}.L^{d} construct behaved very similarly to QL9. B₂m^{b}.L^{d} in that 22% of the surface MCMV. B₂m^{b}.L^{d} molecules were detected in the open conformation (FIG. 1A, part c). By contrast, the p29. β₂m^{b}.L^{d} construct exhibited a higher level of expression of the folded conformers and a much lower expression of the open conformers which corresponds to roughly 8% of the surface pool (FIG. 1A, part d). Identical FACS profiles were obtained when a second independent transfection of DLD-1 cells was performed with these constructs (data not shown). Thus, SCT with peptides known to bind better in solution also make more stable single chain molecules. Therefore, it was found that the level and quality of surface expression of non-covalently bound SCT correlates with the affinity of peptide bind to class I.

### Example 2

SCT recognition by T cells and mAb specific for class I/peptide complexes. SLT constructs were tested with the 2C CTL clone to see if they maintained structural integrity as seen by specific T cells. The CTL clone specifically recognizes L^{d}/QL9 complexes (Sykulev et al., 1994). The β₂m^{b}.L^{d} construct expressed on DLD-1 cells were not recognized by 2C T cells unless exogenous QL9 peptide was added (FIG. 1B). By comparison, DLD-1 cells expressing QL9.β₂m^{b}.L^{d} molecules were recognized by 2C T cells in a dose dependent manner, similar to 2C T cell recognition of DLD-1 cells expressing the β₂m^{b}.L^{d} construct when treated with exogenous QL9 peptide. Similar recognition by L^{d}/MCMV specific T cells was seen with the DLD-1 cells transfected with the MCMV.β₂m^{b}.L^{d} construct (data not shown). Thus SCTs function as targets for antigen-specific T cells.

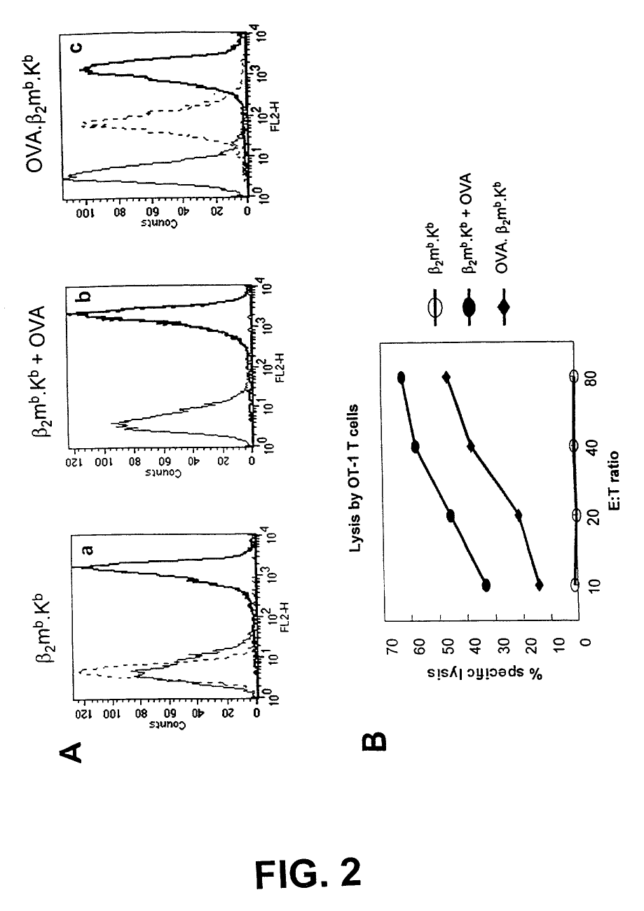

SCT constructs were also prepared containing a K^{b} heavy chain. K^{b} was chosen because it is a prototypical class I molecule that has been used extensively for structure-function analyses. Furthermore, an mAb (25D-1.16) is available that specifically recognizes K^{b}+ the ovalbumin derived SIINFEKL peptide (OVA) (Porgador et al. 1997). This reagent allowed the K^{b}/OVA complexes to be monitored serologically. Thus, a new construct, OVA.β₂m^{b}.K^{b}, was made by replacing the sequence encoding the p29 peptide and L^{d} heavy chain from p29.β₂m^{b}.L^{d} with sequence encoding the OVA peptide and the K^{b} heavy chain. A corresponding β₂m^{b}.K^{b} construct (β₂m^{b}+15 residue linker+K^{b}) was made for comparison. These constructs were transfected into mouse L cells co-expressing ICAM-1 (LM1.8) or DLD-1. The FACS profiles of the LM1.8 transfectants are shown in FIG. 2A. When stained with anti-K^{b} mAb B8-24-3 that is conformationally sensitive but not peptide specific, both constructs gave high level of expression. In accordance with its specificity, mAb 25D1.16 was unreactive with the β₂m^{b}.K^{d} construct unless exogenous OVA peptide was provided (Porgador et al. 1997). By contrast, the OVA.β₂m^{b}.K^{b} construct was reactive with mAb 25D-1.16. This could explain the relatively low level of 25D-1.16 expression. In parallel, the integrity of the OVA.β₂m^{b}.K^{b} construct was also tested by T cell recognition. In this case, K^{b}/OVA specific T cells derived from OT-1 transgenic mice were used (Hogquist et al., 1994). As shown in FIG. 2B, the OVA.β₂m^{b}.K^{d} transfectants were lysed by these OT-1 derived T cells. Thus, the SCT made with both L^{d} and K^{b} are capable recognition by peptide specific T cells. In addition, the K^{b}/OVAN SCT can be detected by an mAb specifically recognizing this particular class I/peptide combination.

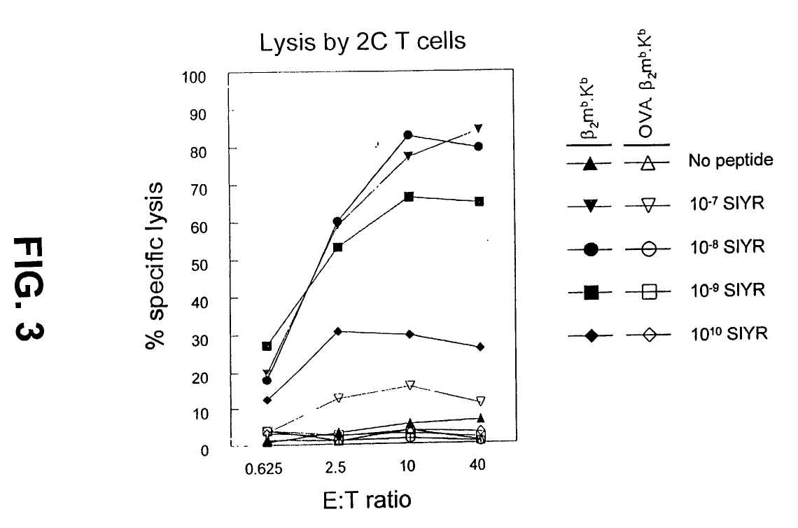

Accessibility of SCT to loading with exogenous peptide. To assess the stability of the covalent peptide which is anchored in the peptide binding groove, peptide competition assays were performed. In this assay, the relative accessibility of the OVA.β₂m^{b}.K^{b} construct to a different K^{b} ligand was monitored. To do this, the 2C CTL clone was again utilized because it also recognizes K^{b}/SIYR complex. SIYR is a synthetic peptide identified from a peptide library (Udaka et al., 1996) and has been reported to be as avid a K^{b} binder as is SIINFEKL (Tallquist et al., 1998). When LM1.8-β₂m^{b}.K^{b} or LM1.8-OVA.β₂m^{b}.K^{b} transfectants were compared as targets for 2C T cells after overnight incubation with graded doses of SIYR peptide (FIG. 3), the OVA.β₂m^{b}.K^{b} construct was completely resistant to displacement by exogenous SIYR peptide at a concentration as high as 10⁻⁷M. Contrary to this, there was significant lysis of LM1.8-β₂m^{b}.K^{b} transfectants at a concentration as low as 10⁻¹⁰M. This finding suggests that the OVA.β₂m^{b}.K^{b} construct is more than 1000-fold less accessible to loading by an exogenous peptide of comparable affinity, when compared with the β₂m^{b}.K^{b} constructs loaded with endogenous peptides. Thus, the covalent peptide is stably bound in the SCT peptide binding groove.

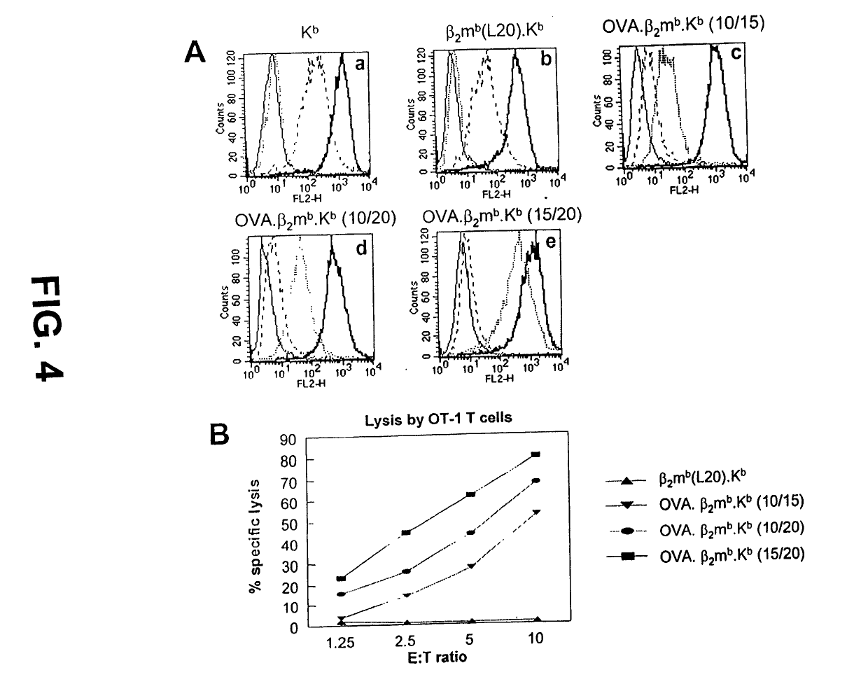

Effect of varying the linker length on the immune recognition of single chain molecules. To test if the single chain construct could be improved further, another set of OVA.β₂m^{b}.K^{b} constructs with longer linkers was created. In addition to varying the linker length, the double mutation R48Q, R50P was introduced into the K^{b} heavy chain to allow the transfer of the epitope detected by the mAb 64-3-7 which recognizes the open conformers (Yu et al., 1999). This epitope tagging (et) has been successfully applied to a number of class Ia and class Ib molecules including K^{b}, K^{d}, HLA-B27 and H2-M3, and found to remain specific for open conformers of the epitope tagged molecule without altering peptide binding specificity (Myers et al. 2000, Yu et al., 1999, Harris et al. 2001; Lybarger et al., 2001). A total of three constructs which were named OVA.β₂m^{b}.K^{b} followed by a bracket indicating the length of the two linkers were made. Thus, for example, OVA.β₂m^{b}.K^{b} (10/15) has a 10 residue linker between the OVA peptide and the β₂m and a 15 residue linker between β₂m^{b} and the K^{b} heavy chain. The other two linker combinations were 10/20 and 15/20. These constructs were compared by flow cytometry to unattached K^{b} or β₂m^{b} (L20).K^{b} (20 residue linker between β₂m and K^{b}) molecules. As shown in FIG. 4A, all of these constructs gave rise to high levels of expression of folded K^{b} (B8-24-3⁺) on LM1.8 cells. However, when examined for the presence of open conformers (64-3-7⁺), only the K^{b} (part a) and β₂m^{b} (L20).K^{b} (part b) constructs expressed an appreciable amount (>10% when expressed as a fraction of the sum of B8-24-3 and 64-3-7 fluorescence intensity). Furthermore, the open conformers associated with the β₂m^{b} (L20).K^{b} construct all but disappeared upon exogenous feeding with the OVA peptide (data not shown) thus reaffirming their “peptide-empty” nature. In stark contrast, the other three transfectants, namely, LM1.8-OVA.β₂m^{b}.K^{b} (10/15), LM1.8-OVA.β₂m^{b}.K^{b} (10/20) and LM1.8-OVA.β₂m^{b}.K^{b} (15/20) expressed less than 1% open conformers. Thus, the covalent OVA peptide must be able to occupy the K^{b} peptide binding groove virtually all the time. When mAb 25D-1.16 reactivity was compared, it was apparent that the linker combination of (15/20) was significantly better than the other two combinations. In parallel with the FACS profiles, the recognition by OT-1 derived T cells was also the best for the transfectants (FIG. 4B). Thus increasing the length of the flexible linkers results in improved recognition of the OVA.β₂m^{b}.K^{b} construct by both the mAb 25D1.16 and OT-1 T cells. This improved recognition with longer linkers in SCT could reflect better peptide positioning and/or reduced steric hindrance for TCR and Ab interaction. All subsequent experiments were preformed using OVAP₂m.^{b}.K^{b} (15/20) molecules with such optimal linkers.

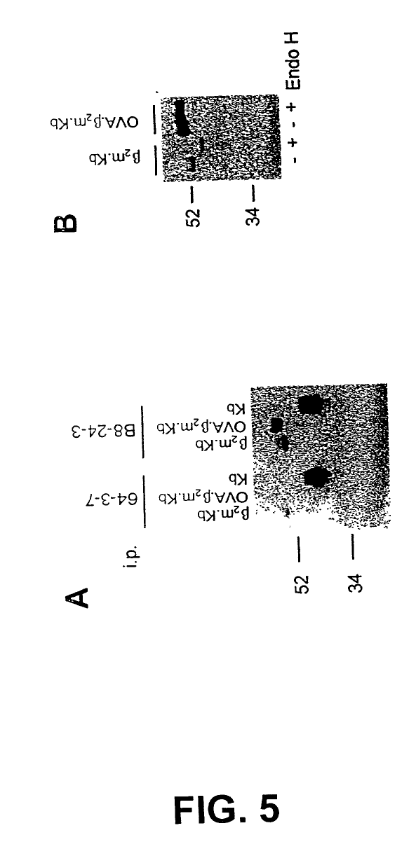

Biochemical integrity of the SCT To examine whether all of the components of the SCT remain intact at the cell surface (FIG. 3), biochemical analyses were performed to compare K^{b}, β₂m.K^{b}, and OVA.β₂m.K^{b}. Each of these molecules was immunoprecipitated from respective L cell transfectants and immunobloted to compare the relative molecular weights of all three K^{b} constructs. As shown in FIG. 5A, mAb 64-3-7 (specific for open heavy chains) precipitated high levels of K^{b}, but low to undetectable amounts of β₂m.K^{b} and OVA.β₂m.K^{b}. By contrast, B8-24-3 (specific for folded K^{b}) was able to precipitate significant amounts of all three constructs. The differential reactivity with these two mAbs demonstrate that the covalent attachments to K^{b} reduced the levels of open conformers existing at steady-state. In addition, this experiment demonstrated that the β₂m.K^{b} and OVA.β₂m.K^{b} covalent constructs exhibit a slower migration consistent with their expected molecular weights. Indeed, the migration of the OVA.β₂m.K^{b} construct was even slower than β₂m.K^{b}, indicating that the covalent OVA peptide and linker remain attached. No breakdown products were evident, including fragments that would correspond in size to K^{b} heavy chains from which the covalent appendages have undergone proteolysis. These results indicate that the preponderance of the single chain molecules, at steady-state, are structurally intact. The doublet bands seen with these constructs represent Endo H-sensitive (ER-resident) vs. Endo H-resistant (post-ER) (FIG. 5B). The β₂m.K^{b} molecules were predominantly Endo H-sensitive, whereas the OVA.β₂m.K^{b} molecules were predominantly Endo H-resistant. This observation suggests that addition of the covalent peptide facilitates faster ER to Golgi transport.

To demonstrate that the OVA peptide was not undergoing proteolysis from the SCT and then rebinding as an unattached peptide, precipitates were preformed using mAb 25D1.16. To compare OVA.β₂m.K^{b} molecules with K^{b} molecules loaded with non-covalently attached OVA peptide, 25-D1.16 precipitates were also formed with β₂m.K^{b} and K^{b} constructs subsequent to overnight incubation with exogenous OVA peptide. FIG. 5C demonstrates that mAb 25-D1.16 precipitated OVA.β₂m.K^{b}, as well as β₂m.K^{b} and K^{b} molecules after incubation with exogenous OVA peptide. Importantly, the SCT migrated slightly slower than the β₂m.K^{b} construct that was precipitated from cells incubated with exogenous peptide. Thus, these precipitates with mAb 25-D1.16 demonstrate that OVA.β₂m.K^{b} molecules retain covalently attached OVA peptide, rather than rebinding free OVA peptide after proteolysis of the SCT.

Accelerated folding and maturation of SCTs. To test whether direct covalent attachment of either β₂m or peptide/β₂m to the heavy chain increases the efficiency of folding, the maturation kinetics of the various K^{b} constructs were compared using pulse-chase analysis. FIG. 5D illustrates that newly synthesized single chain molecules do, indeed, mature more quickly than K^{b} alone. This was apparent both in terms of initial peptide-induced folding (revealed by a loss of 64-3-7 reactivity) and ER to Golgi transport (acquisition of Endo H resistance). Approximately one-half of the K^{b} molecules were Endo H-resistant after 90 minutes, whereas virtually all of the SCTs were resistant at this time point (see mAb B8-24-3 precipitates). Furthermore, addition of the covalent OVA peptide appeared to enhance folding to a greater extent than addition of β₂m alone, since the 64-3-7⁺conformers of OVA.β₂m.K^{b} were lost more rapidly than the 64-3-7⁺β₂m.K^{b} molecules. Taken together, these data indicate that, by covalently appending all of the subunits required for full assembly, class I molecules can assume a folded conformation and traffic from the ER with high efficiency. These findings evidencing that the kinetics of assembly with β₂m and peptide contribute to the overall rate of class I maturation and ER to Golgi transport.

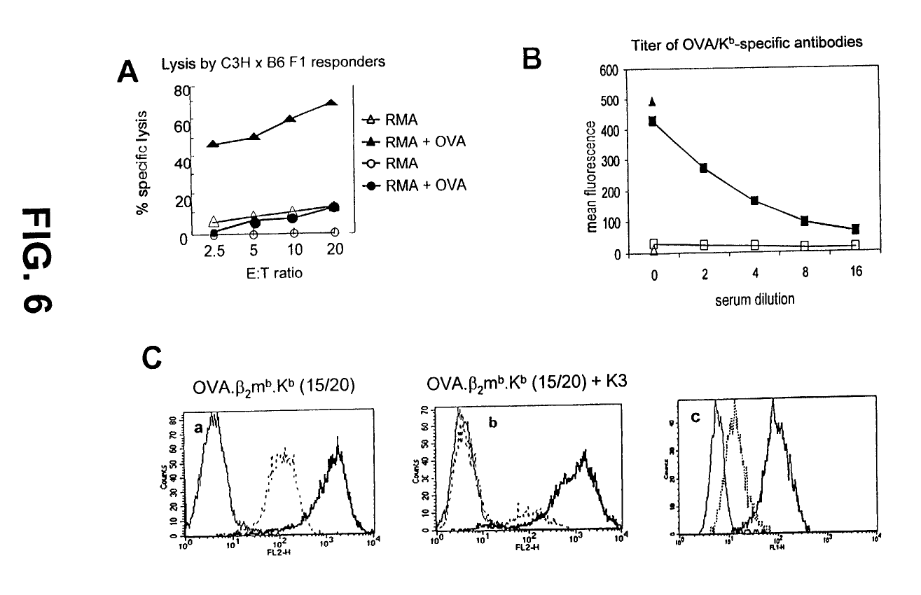

Immunogenicity of SCTs. To test the ability of the single chain class I molecule to generate a T cell response, the ability of LM1.8 (H2^{k}) cells expressing OVA.β₂m^{b}.K^{b} and β₂m^{b}.K^{b} fed exogenous OVA peptide (10⁻⁴M) to induce K^{b}/OVA specific T cell in vitro was compared. For this experiment, responder T cells from [C3H (H2^{k})×B6 (H2^{b})] F1 mice were used that potentially should respond to only K^{b}/OVA complexes presented by either OVA.β₂m^{b}.K^{b} or peptide fed β₂m^{b}.K^{b}. Successful generation of antigen-specific CD8⁺T cells typically requires in vivo priming, intracellular peptide loading or antigen pulsed, purified dendritic cells (Carbone and Bevan, 1989: Mayordomo et al. 1995). However, specific lysis was attainable after just 4 weekly rounds of stimulation splenocytes with cells expressing the OVA. β₂m^{b}.K^{b} construct (data not shown). High levels K^{b}/OVA-specific lysis was observed after 5 weekly rounds of stimulation with this same construct (FIG. 6A, part a). By comparison, after 5 weekly rounds of stimulation with cells expressing the β₂m^{b}.K^{b} construct and fed 10⁻⁴M continuous OVA peptide, little if any K^{b}/OVA-specific lysis was observed (FIG. 6A, part b). Thus the single chain class I construct including peptide is superior in stimulating peptide specific T cells. Given that the OVA.β₂m^{b}.K^{b} construct is more than a 1000 fold less accessible to exogenous peptide than the β₂m.K^{b} construct (FIG. 3C), it is highly unlikely that the OVA.β₂m.K^{b} construct is a more potent stimulator due to the covalent OVA peptide being clipped off and rebinding as a free peptide.

To demonstrate that SCTs retain immune recognition as intact structures in vivo, mice were vaccinated with DNA encoding OVA.β₂m^{b}.K and then tested for antibody production. DNA vaccination was preformed using allogeneic BALB/c mice to eliminate the possibility of cross presentation of the OVA peptide on self Kb molecules. After only two injections of DNA, 2/6 BALB/c recipients made significant antibodies (titer 1:16). These antibodies were found to be predominantly Kb/ova specific, since they did not detect Kb loaded with endogenous peptides (FIG. 6B), or an irrelevant peptide (data not shown). Together these findings demonstrate that the OVA.β₂m^{b}.K^{b} single chain construct is highly immunogenic due to its capacity to remain covalently attached and to stimulate peptide-specific, class I restricted, CD8 T cells and antibodies.

Resistance of SCT to down regulation by the K3 protein of γ-HV68. To test the resistance of a single chain construct to down regulation by a virus protein, the K3 protein encoded by murine γ-HV68 was tested. In a recent report γ-HV68 K3 expression was shown to severely reduce K^{b} and D^{b} expression by inducing a rapid turnover of immature (EndoH-sensitive) class I molecules (Stevenson et al., 2000). To test whether single chain class I molecules were also susceptible to K3 mediated down regulation, a K3 Cdna was stably introduced into the LM1.8 transfectant expressing the OVA.β₂m^{b}.K^{b} construct. As can be seen in FIG. 6B (parts a and b), the introduction of K3 almost completely shut down the endogenous D^{k} expression while the OVA.β₂m^{b}.K^{b} expression remained largely unscathed. As a control, stable expression of K3 was found to sharply reduce the amount of endogenous K^{b} (lacking any attachments) expressed on the cell surface of B6/WT-3 cells (FIG. 6B, part c). Thus, the OVA.β₂m^{b}.K^{b} single chain class I construct effectively escapes K3-mediated down regulation.

### REFERENCES

Ahn, K., Gruhler, A., Galocha, B., Jones, T. R., Wietz, E. J. H. J., Ploegh, H. L., Peterson, P. A., Yang, Y., and Früh, K. (1997). The ER-luminal domain of the HCMV glycoprotein US6 inhibits peptide translocation by TAP. Immunity 6, 613-621.

Balendiran, G. K., Solheim, J. S., Young, A. C. M., Hansen., T. H., Nathenson, S. G., and Sacchettini, J.C. (1997). The three-dimensional structure of an H-2L^{d}-peptide complex explains the unique interaction of L^{d} with beta-2-microglobulin and peptide. Proc. Natl. Acad. Sci. USA 94, 6880-6885.

Bennett, E. M., Bennink, J. R., Yewdell, J. W., and Brodsky, F. M. (1999). Adenovirus E19 has two mechanisms for affecting class I MHC expression. J. Immunol. 162, 5049-5052.

Bicknell, D.C., Rowan, A., and Bodmer, W. F. (1994). Beta 2-microglobulin gene mutations: a study of established colorectal cell lines and fresh tumors. Proc. Natl. Acad. Sci. USA 91, 4751-4756.

Carbone, F. R., and Bevan, M. J. (1989). Induction of ovalbumin-specific cytotoxic T cells in vivo. J. Exp. Med. 169: 603-12.

Chung, D. H., Dorfman, J., Plaksin, D., et al. (1999). NK and CTL recognition of a single chain H-2D^{d} molecule: distinct sites of H-2D^{d} interact with NK and TCR. J. Immunol. 163, 3699-3708.

Corr, M., Boyd, L. F., Frankel, S. R., Kozlowski, S., Padlan, E. A., and Margulies, D. H. (1992). Endogenous peptide of a soluble major histocompatility complex class I molecule, H-2L^{d}ₛ:sequence motif, quantitative binding, and molecular modeling of the complex. J. Exp. Med. 176, 1681-1692.

Degen, E., and Williams, D. B. (1991). Participation of novel 88-kD protein in the biogenesis of murine class I histocompatibility molecules. J. Cell Biol. 112, 1099-1115.

Dela Cruz, C. S., Tan, R., Rowland-Jones, S. L., and Barber, B. H. (2000). Creating HIV-1 reverse transcriptase cytotoxic T lymphocyte target structures by HLA-A2 heavy chain modifications. Int. Immunol.12, 1293-1302.

Dexter, D. L., Barbosa, J. A., and Calabresi, P. (1979). N,N-dimethyformamide-induced alteration of cell culture characteristics and loss of tumorigenicity in cultured human colon carcinoma cells. Cac. Res. 39, 1020-1025.

Fremont, D. H., Matsumura, M., Stura, E. A., Peterson, P.A., and Wilson, I. A., (1992). Crystal structures of two viral peptides in complex with murine MHC class I H-2K^{b}. Science 257, 919-927.

Fremont, D. H., Strura, E. A., Masazumi, M., Peterson, P. A., and Wilson, I. A., (1995) Crystal structure of an H-2K^{b}-ovalbumin peptide complex reveals the interplay of primary and secondary anchor positions in the major histocompatibility complex binding groove. Proc. Natl. Acad. Sci. USA 92, 2479-2483.

Früh, K., Ahn, K., Djaballah, H., Sempë, P., van Endert, P. M., Tampë, R., Peterson, P. A., and Yang. Y. (1995). A viral inhibitor of peptide transporters for antigen presentation. Nature 375, 415-418.

Gorka, J. D., McCourt, D. W., and Schwartz, B. D. (1989). Automated synthesis of a C-terminal photoprobe using combined Fmoc and t-Boc synthesis strategies on a single automated peptide synthesizer. Pept. Res. 2, 367-380.

Grandea, A. G., and Van Kaer, L. (2001). Tapasin: and ER chaperone that controls MHC class I assembly with peptide. Trends Immunol. 22, 194-9.

Hansen, T., Balendiran, G., Solheim, J., Ostrov, D., and Nathenson, S. (2000). Structural features of MHC class I molecules that might facilitate alternative pathways of presentation. Immunol. Today 21, 83-88.

Harris, M. R., Lybarger, L., Myers, N. B., Hilbert, C., Solheim, J. C., Hansen, T. H., and Yu, Y. Y. L. (2001). Interactions of HLA-B27 with the peptide loading complex as revealed by heavy chain mutations. Int Immunol., in press.

Hengel, H., Brune, W., and Koszinowski, U. H. (1998). Immune evasion by cytomegalovirus—survival strategies of a highly adapted opportunist. Trends Microbiol. 6, 190-197.

Hill, A., Jugovic, P., York, L., Russ, G., Bennink, J., Yewdell, J., Ploegh, H., and Johnson, D. (1995). Herpes simplex virus turns off the TAP to evade host immunity. Nature 375, 411-415.

Hogquist, K. A., Jameson, S.C., Heath, W. R., Howard, J. L., Bevan. M. J., and Carbone, F. R. (1994). T cell receptor antagonist peptides induce positive selection. Cell 76, 17-17.

Ignatowicz, L., Kappler, J., and Marrack, P. (1996). The repertroire of T cells shaped by a single MHC/peptide ligand. Cell 23, 521-529.

Jaulin, C., Romero, P., Luescher, I. F., Casanova, J.-L., Prochnicka-Chalufour, A., Langlade-Demoyen, P., Maryanski, J. L., and Kourilsky, P. (1992). Most residues on the floor of the antigen binding site of the class I MHC molecule H-2K^{d} influence peptide presentation. Int. Immunol. 4, 945-953.

Koller, B. H., Marrack, P., Kappler, J. W., and Smithies, O. (1990) normal development of mice deficient in β2m, MHC class I proteins, and CD8+ T cells. Science 248, 1227-1230.

Levitskaya, J., Coram, M., Levitsky, V., Imreh, S., Steigerwald-Mullen, P. M., Klein, G., Kurilla, M. G., and Masucci, M. G. (1995). Inhibition of antigen processing by the internal repeat region of the Epstein-Barr virus nuclear antigen-1. Nature 375, 685-688.

Lie, W.-R., Myers, N. B., Connolly, J. M., Gorka, G., Lee, D. R. and T. H. Hansen. (1991). The specific binding of peptide ligand to L^{d} class I major histocompatibility complex molecules determines their antigen structure. J. Exp. Med. 173, 449-459.

Long, E. O., Rosen-Bronson, S., Karp, D. R., Malnati, M., Sekaly, R. P., and Jaraquemada, D. (1991). Efficient cDNA expression vectors for stable and transient expression of HLA-DR in transfected fibroblast and lymphoid cells. Human Immunol. 31, 229-235.

Lybarger, L., Yu, Y. Y. L., Chun, T., Wang, C.-R., Grandea III, A. G., Van Kaer, L., and Hansen, T. H. (2001). Tapasin enhance peptide-induced expression of H2-M3 molecules but is not required for the retention of open conformers. J. Immunol. 167: 2097-2105.

Madden, D. R., Gorga, J. C., Strominger, J. L., and Wiley, D. C. (1992). The three-dimensional structure of HLA-B27 at the 2.1 A resolution suggests a general mechanism for tight peptide binding. Cell 70,1035-1048.

Mage, M. G., Lee, L., Ribaudo, R. K., Corr, M., Kozlowski, S., McHough, L., and Margulies, D. H. (1992). A recombinant, single-chain class I major histocompatibility complex molecule with biological activity. Proc. Natl. Acad. Sci. USA 89; 10658-10662.

Matsumura, M., Fremont, D. H., Peterson, P. A., and Wilson, I. A. (1992). Emerging principles for the recognition of peptide antigens by MHC class I molecules. Science, 927-934.

Merrifield, R. B. (1963). Solid phase peptide synthesis. I. The synthesis of a tetrapeptide. J. Am. Chem. Soc. 85, 2149-2154.

Messaoudi, I., LeMaoult, J., and Nikolic-Zugic, J. (1999). The mode of ligand recognition by two peptide:MHC class I-specific monoclonal antibodies. J. Immunol. 163, 3286-3294.

Miller, D. M., and Sedmak, D. D. (1999). Viral effects on antigen processing. Current Opinion in Immunol. 11, 94-99.

Mottez, E., Langlade-Demoyen, P., Gournier, H., Martinon, F., Maryanski, J., Kourilsky, P., and Abastado, J. P. (1995) Cells expressing a major histocompatibility complex class I molecule with a single covalently bound peptide are highly immunogenic. J. Exp. Med. 181, 493-502.

Myers, N. B., Harris, R. M.; Connolly, J. M., Lybarger, L., Yu, Y. Y. L. and Hansen, T. H. (2000). K^{b}, K^{d}, and L^{d} molecules share common tapasin dependencies as determined using a novel epitope tag. J. Immunol. 165, 5656-5563.

Perarnau, B., Saron, M.-F., Martin, B. R. S., Bervas, N., Ong, H., Soloski, M. J., Smith A. G., Ure, J. M., Gairin, J. E., and Lemonnier, F. A. (1999) Single H2K^{b}, H2D^{b} and double H2K^{b}D^{b} knockout mice: peripheral CD8⁺ T cell repertoire and anti-LCMV cytolytic responses. Eur J. Immunol. 29:110

Pamer, E., and Cresswell, P. (1998). Mechanisms of MHC class I-restricted antigen processing. Annu. Rev. Immunol. 16, 323-358.

Ploegh, H. L. (1998). Viral strategies of immune evasion. Science 280, 248-253.

Porgador, A., Yewdell, J. W., Deng, Y., Bennink, J. R., and Germain, R. N. (1997). Localization, quantitation, and in situ detection of specific peptide-MHC class I complexes using a monoclonal antibody. Immunity 6, 715-726.

Pretell, J., Greenfield, R. S., and Tevethia, S. S. (1979). Biology of simian virus 40 (SV40) transplantation antigen (TrAg) V. In vitro demonstration of SV40 TrAg in SV40 infected nonpermissive mouse cells by the lymphocyte mediated cytotoxicity assay. Virology 97, 32-41.

Raposo, G., van Santen, H. M., Leijendekker, R., Geuze, H. J., and Ploegh, H. L. (1995). Misfolded major histocompatibility complex class I molecules accumulate in an expanded ER-Golgi intermediate compartment. J. Cell Biol. 131, 1403-1419.

Reddehase, M. J., Rothbard, J. B., and Koszinowski, U. H. (1989). A pentapeptide as minimal antigenic determinant for MHC class I-restricted T lymphocytes. Nature 33 7, 651-653.

Seliger, B., Maeurer, M. J., and Ferrone, S. (2000). Antigen-processing machinery breakdown and tumor growth. Immunol. Today 21, 455-464.

Smith, J. D., Myers, N. B., Gorka, J., and Hansen, T. H. (1992). Disparate interaction of peptide ligand with nascent versus mature class I major histocompatibility complex molecules: comparisons of peptide binding to alternative forms of L^{d} in cell lysates and at the cell surface. J. Exp. Med. 175, 191-202.

Smith, J. D., Myers, N. B., Gorka, J. and Hansen, T. H. (1993). Model for the in vivo assembly of nascent L^{d} class I molecules and for the expression of unfolded L^{d} molecules at the cell surface. J. Exp. Med. 178, 2035-2046.

Spiliotis, E. T., Osorio, M., Zuniga, M. Z. and Edidin, M. (2000). Selective export of MHC molecules from the ER after their dissociation from TAP. Immunity 13, 841-851.

Stevenson, P. G., Efstathiou, S., Doherty, P C., and Lehner, P. J. (2000). Inhibition of MHC class I-restricted antigen presentation by γ2-herpesviruses. Proc. Natl. Acad. Sci. USA 97, 8455-8460.

Stryhn, A., Pedersen, L. O., Holm, A., and Buus, S. (2000). Longer peptide can be accommodated in the MHC class I binding site by a protrusion mechanism. Eur. J. Immunol. 30, 3089-3099.

Sykulev, Y., Brunmark, A., Tsomides, T. J., et al. (1994). High affinity reactions between antigen-specific T-cell receptors and peptides associated with allogeneic and syngeneic major histocompatibility complex class I proteins. Proc. Natl. Acad. Sci. USA 91, 11487-11491.

Tallquist, M. D., Weaver, A. J., and Pease, L. R. (1998). Degenerate recognition of alloantigenic peptides on a positive-selecting class I molecule. J. Immunol. 160, 802-809.

Toshitani, K., Braud, V., Browning, M. J., Murry, N., and McMichael, A. J. (1996). Expression of a single-chain HLA class I molecule in a human cell line: presentation of exogenous peptide and processed antigen to cytotoxic T lymohocytes. Proc. Natl. Acad. Sci. 93, 236-240.

Townsend, A., Elliot, T., Cerundolo, V., Foster, L., Barber, B., and Tse, A. (1990). Assembly of MHC class I molecules analysed in vitro. Cell 62, 285-295.

Udaka, K., Tsomides, T. J., and Eisen, H. N. (1992). A naturally occurring peptide recognized by alloreactive CD8⁺ cytotoxic T lymphocytes in association with a class I MHC protein. Cell, 69, 989-998.

Udaka, K., Weismüller, K.-H., Kienle, S., Jung, G., and Walden, P. (1996). Self-MHC-restricted peptides restricted by an alloreactive T lymphocyte clone. J. Immunol. 157, 670-678.

Uger, R. A., and Barber, B. H. (1998). Creating CTL targets with epitope-linked to β₂-microglobulin constructs. J. Immunol. 160, 1598-1605.

Uger, R. A., Chan, S. M., and Barber, B. H. (1999). Covalent linkage of β₂-microglobulin enhances the MHC stability and antigenicity of suboptimal CTL epitopes. J. Immunol. 162, 6024-6028.

Van Kaer, L., Ashton-Rickardt, P. G., Ploegh, H. L. and Tonegawa, S. (1992). TAP1 mutant mice are deficient in antigen presentation, surface class I molecules, and CD4⁻8⁺ T cells. Cell 71:1205-1214.

White, J., Crawford, F., Fremont, D., Marrack, P., and Kappler, J. (1999). Soluble class I MHC with β₂-microglobulin covalently linked peptides: specific binding to a T cell hybridoma. J. Immunol. 162, 2671-2676.

York, I. A., Roop, C., Andrews, D. W., Riddell, S. R., Graham, F. L. and Johnson, D. C. (1994). A cytosolic herpes simplex virus protein inhibits antigen presentation to CD8⁺ T lymphocytes. Cell 77, 525-535.

Yu, Y. Y. L., Myers, N. B., Hilbert, C. M., Harris, M. R., Balendiran, G. K. and Hansen, T. H. (1999). Definition and transfer of a serological epitope specific for peptide empty forms of MHC class I. Int. Immunol. 11, 1897-1906.

Zeidler, R., Eissner, G., Meissner, P., Uebel, S, Tampe, R., Lazis, S., Hammerschmidt, W. (1997). Downregulation of TAP 1 in B lymphocytes by cellular and Epstein-Barr virus-encoded interleukin 10. Blood 90, 2390-2397.

Zijlstra, M, Bix, N., Simistier, N., Loring, J., Raulet, D., and Jaenish, R. (1990). β₂-microglobulin deficient mice lack CD4-8⁺ cells. Nature 344, 743-746.

We claim:

1. A recombinant DNA molecule comprising a DNA sequence encoding a single chain trimer of a mature MHC molecule, the single chain trimer comprising in sequence:
(1) a peptide ligand segment;
(2) a first linker;
(3) a β₂m segment;
(4) a second linker; and
(5) a class I heavy chain segment,
wherein the peptide ligand segment has a carboxy end, the β₂m segment has amino and carboxy ends, and the heavy chain segment has an amino end, and wherein the peptide ligand segment is covalently linked via its carboxy end to the amino end of the β₂m segment by the first linker, and wherein the β₂m segment is covalently linked via its carboxy end to the amino end of the heavy chain segment by the second linker.

2. The recombinant DNA molecule of claim 1 wherein the class I heavy chain segment is comprised of a HLA-A, HLA-B, HLA-C, 1^{a}, 1^{b}, H-2-K, H-2-D^{d} or H-2-L^{d} heavy chain.

3. The recombinant DNA molecule of claim 1 wherein the class I heavy chain segment contains a mutated conserved residue.

4. The recombinant DNA molecule of claim 3 wherein the tyrosine at position 84 is mutated.

5. The recombinant DNA molecule of claim 1 wherein the first linker is comprised of at least 10 amino acid residues.

6. The recombinant DNA molecule of claim 5 wherein the first linker is comprised of at least 15 amino acid residues.

7. The recombinant DNA molecule of claim 6 wherein the first and second linkers are comprised of at least about 80 percent glycine, alanine or serine residues.

8. The recombinant DNA molecule of claim 1 wherein the second linker is comprised of least 15 amino acid residues.

9. The recombinant DNA molecule of claim 8 wherein the second linker is comprised of at least 20 amino acid residues.

10. The recombinant DNA molecule of claim 9 wherein the first and second linkers are comprised of at least about 80 percent glycine, alanine or serine residues.

11. The recombinant DNA molecule of claim 1 wherein the peptide ligand segment comprises an antigenic peptide.

12. The recombinant DNA molecule of claim 11 wherein the peptide ligand segment contains from about 4 to 30 amino acid residues.

13. The recombinant DNA molecule of claim 12 wherein the peptide ligand segment contains from about 6 to 20 amino acid residues.

14. The recombinant DNA molecule of claim 13 wherein the peptide ligand segment contains from about 8 to 12 amino acid residues.

15. The recombinant DNA molecule as claimed in claim 1, wherein the DNA sequence is contained in a vector.

16. A host transformed with the vector of claim 15.

17. A recombinant DNA molecule comprising a DNA sequence encoding a single chain trimer of a mature MHC molecule, the single chain trimer comprising in sequence:
(1) an antigenic peptide ligand segment containing from about 4 to 30 amino acid residues;
(2) a first linker comprising at least 10 amino acid residues;
(3) a β₂m segment;
(4) a second linker comprising at least 15 amino acid residues; and
(5) a heavy chain segment comprising an HLA-A, HLA-B, HLA-C, 1^{a}, 1^{b}, H-2-K, H-2-D^{d} or H-2-L^{d} heavy chain,
wherein the peptide ligand segment has a carboxy end, the β₂m segment has amino and carboxy ends, and the heavy chain segment has an amino end, and wherein the peptide ligand segment is covalently linked via its carboxy end to the amino end of the β₂m segment by the first linker, and wherein the β₂m segment is covalently linked via its carboxy end to the amino end of the heavy chain segment by the second linker.

18. The recombinant DNA molecule of claim 17 wherein the class I heavy chain segment contains a mutated conserved residue.

19. The recombinant DNA molecule of claim 18 wherein a tyrosine at position 84 is mutated.

20. The recombinant DNA molecule of claim 17 wherein the first linker comprises at least 15 amino acid residues and the second linker comprises at least 20 amino acid residues.

21. The recombinant DNA molecule of claim 17 wherein the peptide ligand segment contains from about 8 to 12 amino acid residues.

22. A class I heavy chain containing a mutated conserved residue.

23. The class I heavy chain of claim 22 wherein the tyrosine at position 84 is mutated.

24. The recombinant DNA molecule as claimed in claim 17, wherein the DNA sequence is contained in a vector.

25. A host transformed with the vector of claim 24.

26. A single chain trimer of a mature Class I MHC molecule comprising
(1) a peptide ligand segment having a carboxy end;
(2) a first linker;
(3) a β₂M segment having amino and carboxy ends;
(4) a second linker; and
(5) a class I heavy chain segment having an amino end, wherein the β₂m segment and the heavy chain segment are encoded by a mammalian Class I MHC locus, wherein the carboxy end of the peptide ligand segment is covalently linked to the amino end of the β₂m segment via a first flexible peptide linker, and wherein the carboxy end of the β₂m segment is covalently linked to the amino end of the class I heavy chain segment via a second flexible peptide linker.

27. The single chain trimer of claim 26 wherein the class I heavy chain segment is comprised of an HLA-A, HLA-B, HLA-C, 1^{a}, 1^{b}, H-2-K, H-2-D^{d} or H-2-L^{d} heavy chain.

28. The single chain trimer of claim 26 wherein the class I heavy chain segment contains a mutated conserved residue.

29. The single chain trimer of claim 28 wherein the tyrosine at position 84 is mutated.

30. The single chain trimer of claim 26 wherein the first linker comprises at least 10 amino acid residues.

31. The single chain trimer of claim 30 wherein the first linker comprises at least 15 amino acid residues.

32. The single chain trimer of claim 31 wherein at least about 80 percent of the linkers comprise glycine, alanine or serine residues.

33. The single chain trimer of claim 32 wherein the second linker comprises at least 15 amino acid residues.

34. The single chain trimer of claim 33 wherein the second linker comprises at least 20 amino acid residues.

35. The single chain trimer of claim 34 wherein at least about 80 percent of the linkers comprise glycine, alanine or serine residues.

36. The single chain trimer of claim 26 wherein the peptide ligand comprises an antigenic peptide.

37. The single chain trimer of claim 36 wherein the peptide ligand contains from about 4 to 30 amino acid residues.

38. The single chain trimer of claim 37 wherein the peptide ligand contains from about 6 to 20 amino acid residues.

39. The single chain trimer of claim 38 wherein the peptide ligand contains from about 8 to 12 amino acid residues.

40. A single chain trimer of a mature Class I MHC molecule comprising:
(1) an antigenic peptide ligand segment containing from about 4 to 30 amino acid residues and having a carboxy end
(2) a first linker comprising at least 10 amino acid residues;
(3) a β₂m segment having amino and carboxy ends;
(4) a second linker comprising at least 10 amino acid residues; and
(5) a heavy chain segment comprising an HLA-A, HLA-B, HLA-C, 1^{a}, 1^{b}, H-2-K, H-2-D^{d}, and H-2-L^{d} heavy chain having an amino end, wherein the β₂m segment and the heavy chain segment are encoded by a mammalian Class I MHC locus, wherein the carboxy end of the peptide ligand segment is covalently linked to the amino end of the β₂m segment via a first flexible peptide linker, and wherein the carboxy end of the β₂m segment is covalently linked to the amino end of the class I heavy chain segment via a second flexible peptide linker.

41. The single chain trimer of claim 40 wherein the class I heavy chain segment contains a mutated conserved residue.

42. The single chain trimer of claim 41 wherein a tyrosine at position 84 is mutated.

43. The single chain trimer of claim 42 wherein the first linker comprises at least 15 amino acids and the second linker comprises at least 15 amino acids.

44. The single chain trimer of claim 40 wherein the peptide ligand contains from about 8 to 12 amino acid residues.

45. A mutein of a class I heavy chain molecule having an amino acid other than tyrosine substituted at position 84.
